(19) United States
(12) Patent Application Publication
(10) Pub. No.: US 2023/0056796 A1
(43) Pub. Date: Feb. 23, 2023

(54) COMPOSITION COMPRISING A CATIONIC CELLULOSE-BASED POLYMER, A SULFONIC ANIONIC POLYMER AND A FIXING POLYMER

(71) Applicant: L'OREAL, Paris (FR)

(72) Inventors: Guillaume RONCHARD, Saint-Ouen (FR); Virginie SOULIE, Saint-Ouen (FR)

(21) Appl. No.: 17/787,092

(22) PCT Filed: Dec. 18, 2020
(86) PCT No.: PCT/EP2020/087221

§ 371 Date: Jun. 17, 2022

(30) Foreign Application Priority Data
Dec. 20, 2019 (FR) FR1915114

(51) Int. Cl. A61K 8/73; A61K 8/81; A61K 8/41; A61Q 5/02

(52) U.S. Cl. CPC A61K 8/731; A61K 8/8188; A61K 8/8158; A61K 8/416; A61Q 5/02

(57) ABSTRACT

The present invention relates to a composition, which is preferably aqueous, comprising at least one cationic cellulose-based polymer, at least one sulfonic anionic polymer and at least one fixing polymer. The present invention also relates to a cosmetic treatment process, in particular a process for styling keratin fibers, in particular human keratin fibers such as the hair, using this composition. Finally, the present invention relates to the use of this composition for the cosmetic treatment of keratin fibers, in particular human keratin fibers such as the hair, and in particular for hair styling, i.e. shaping and/or fixing the head of hair.

The present invention relates to a composition, which is preferably aqueous, comprising at least one cationic cellulose-based polymer, at least one sulfonic anionic polymer and at least one fixing polymer.

The present invention also relates to a cosmetic treatment process, in particular a process for styling keratin fibers, in particular human keratin fibers such as the hair, using this composition.

Finally, the present invention relates to the use of this composition for the cosmetic treatment of keratin fibers, in particular human keratin fibers such as the hair, and in particular for hair styling, i.e. shaping and/or fixing the head of hair.

Styling products are normally used to construct and structure the hairstyle and to give it hold. They are usually in the form of lotions, gels, mousses, creams or sprays. These compositions generally comprise one or more film-forming polymers, or “fixing polymers”, which allow the formation of a coating film on the hair and thus ensure the hold of the hairstyle and/or the formation of micro-bonds between the individual hairs, thus ensuring the fixing of the head of hair.

These compositions are generally applied to wet hair, which is shaped before performing blow drying or drying.

To obtain satisfactory and long-lasting fixing power, it is known practice to incorporate into styling products polymers with very high fixing power, and/or to increase the concentration of fixing polymer. However, the use of such extremely fixing products causes a certain number of drawbacks.

Although the objective of these products is to ensure the fixing and the hold of the hairstyle over time, they generally have a tendency to make the hairstyle rigid, in particular producing a “helmet effect”, often poorly perceived by users.

The head of hair, thus made rigid, exhibits a dry and rough feel which is not greatly appreciated by consumers.

Moreover, when the styling products are in the form of mousses, they may have more or less firm textures with limited hold in the hand. Conversely, the textures of gels may be greasy or tacky and may prove to be difficult to apply to the head of hair.

Thus, there is a real need to develop styling compositions which do not exhibit the abovementioned drawbacks, i.e. which make it possible to obtain long-lasting fixing of the hairstyle, with styling effects which persist throughout the day, or even for several days, while preserving a natural and non-rigid appearance on the hairstyle.

There is in particular a need to develop compositions which give good styling properties, in particular in terms of suppleness and volume, while providing a pleasant cosmetic feel, notably a smooth and soft feel, a clear visual appearance, which is neither mattifying nor too shiny. It is also expected to be possible for the roots of the hair to be lifted, and for the head of hair to have more volume.

There is also a need to develop compositions which have a nontacky and non-greasy texture, that are easy to apply.

The aim of the present invention is also to have available a composition which makes it possible to obtain good hair fixing and hold properties, while at the same time preserving a clean and non-tacky feel of the hair and which may optionally be used directly during showering or shampooing.

The applicant has discovered, surprisingly, that the combination of a cationic cellulose-based polymer, a sulfonic anionic polymer and a fixing polymer makes it possible to achieve the objectives set out above; notably that of obtaining a styling composition that is easy to apply and capable of conferring long-lasting fixing on the hairstyle while at the same time preserving a natural and non-rigid appearance, and also a smooth and soft feel.

A subject of the invention is notably a composition comprising:
a) one or more cationic cellulose-based polymers,
b) one or more sulfonic anionic polymers and salts thereof,
c) one or more fixing polymers other than the polymers (a) and the polymers
   the sulfonic anionic polymers being chosen from:
      homopolymers of acrylamidoalkylsulfonic acid and salts thereof,
      sulfonic polyesters,
      amphiphilic polymers of at least one acrylamido-2-methylpropanesulfonic acid monomer (AMPS®) and of at least one ethylenically unsaturated hydrophobic comonomer comprising at least one hydrophobic part containing from 7 to 30 carbon atoms.

The composition of the invention has a pleasant, non-tacky and non-greasy texture, easy to apply to the whole of the head of hair to be styled. It also makes it possible to give the hair suppleness and volume, while at the same time preserving a natural appearance and a smooth and soft feel.

Contrary to the prior art compositions, the head of hair thus styled is held without being rigid, and the styling effects conferred by the composition of the invention are long-lasting throughout the day, or even for several days.

Another subject of the present invention is a process for the cosmetic treatment of keratin fibers, in particular human keratin fibers such as the hair, comprising a step of applying, to said keratin fibers, a composition as defined above, preferably followed by a step of rinsing with water or an aqueous composition.

The present invention also relates to the use of a composition as defined above for styling, that is to say shaping and/or fixing, keratin fibers, in particular human keratin fibers such as the hair.

Other subjects, characteristics, aspects and advantages of the invention will become even more clearly apparent on reading the description and the examples which follow.

In that which follows, and unless otherwise indicated, the limits of a range of values are included in this range, in particular in the expressions “of between” and “ranging from . . . to . . . ”.

Moreover, the expression “at least one” used in the present description is equivalent to the expression “one or more”.

For the purposes of the present invention, the term “polymer” is intended to denote a compound comprising at least two repeating units and in particular at least five repeating units.

Cationic Cellulose-Based Polymer

The composition according to the present invention comprises one or more cationic cellulose-based polymers.

For the purposes of the present invention, the expression “cationic cellulose-based polymer” denotes any non-silicone (not comprising any silicon atoms) cellulose-based polymer containing cationic groups and/or groups that can be ionized into cationic groups and preferably not containing any anionic groups and/or groups that can be ionized into anionic groups.

According to the invention, the term “cellulose-based” polymer means any polysaccharide compound having in its structure at least 20 sequences of glucose residues bonded together via 13-1,4 linkages.

The cellulose-based polymer may be associative, i.e. it may bear in its structure at least one C₈-C₃₀ fatty chain.

Alternatively, the cellulose-based polymer may be non-associative, i.e. not bearing any C₈-C₃₀ fatty chains.

The cationic cellulose-based polymers that may be used preferably have a weight-average molar mass (Mw) of between 5000 and 5×10⁶ approximately and preferably between 10³ and 3×10⁶ approximately.

Among cationic celluloses, mention may more particularly be made of cellulose ethers comprising quaternary ammonium groups optionally modified with groups comprising at least one fatty chain, cationic cellulose copolymers or cellulose derivatives grafted with a water-soluble quaternary ammonium monomer.

The cellulose ether derivatives comprising quaternary ammonium groups are in particular described in FR 1 492 597, and mention may be made of the polymers sold under the name Ucare Polymer JR (JR 400 LT, JR 125 and JR 30M) or LR (LR 400 and LR 30M) by the company Amerchol. These polymers are also defined in the CTFA dictionary as hydroxyethylcelluloses that have reacted with an epoxide substituted by a trimethylammonium group and called Polyquaternium-10.

The cationic cellulose copolymers or celluloses grafted with a water-soluble quaternary ammonium monomer are described in particular in patent U.S. Pat. No. 4,131,576, and mention may be made of hydroxyalkyl celluloses, for instance hydroxymethyl, hydroxyethyl or hydroxypropyl celluloses grafted, in particular, with a methacryloylethyltrimethylammonium, methacrylamidopropyltrimethylammonium or dimethyldiallylammonium salt. The commercial products corresponding to this definition are more particularly the products sold under the names Celquat L 200 and Celquat H 100 by the company National Starch.

Among the quaternized celluloses, mention may in particular be made of quaternized celluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24, or even from 10 to 14, carbon atoms; or mixtures thereof.

Preferably, mention may be made of quaternized hydroxyethylcelluloses modified with groups comprising at least one fatty chain, such as linear or branched alkyl groups, linear or branched arylalkyl groups, or linear or branched alkylaryl groups, preferably linear or branched alkyl groups, these groups comprising at least 8 carbon atoms, in particular from 8 to 30 carbon atoms, better still from 10 to 24, or even from 10 to 14, carbon atoms; or mixtures thereof.

Preferentially, mention may be made of the hydroxyethylcelluloses of formula (Ib):

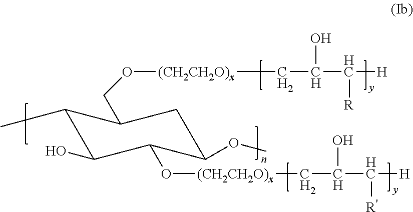

wherein:
R represents an ammonium group RaRbRcN⁺—, Q⁻ wherein Ra, Rb and Rc, which may be identical or different, represent a hydrogen atom or a linear or branched C₁-C₃₀ alkyl, preferably an alkyl, and Q⁻ represents an anionic counterion such as a halide, for instance a chloride or bromide;
R′ represents an ammonium group R′aR′bR′cN⁺—, Q′⁻ wherein R′a, R′b and R′c, which may be identical or different, represent a hydrogen atom or a linear or branched C₁-C₃₀ alkyl, and represents an anionic counterion such as a halide, for instance a chloride or bromide; preferably an alkyl;

it being understood that at least one of the radicals Ra, Rb, Rc, R′a, R′b and R′c represents a linear or branched C₈-C₃₀ alkyl;
n, x and y, which may be identical or different, represent an integer between 1 and 10 000.

Preferably, in formula (Ib), at least one of the radicals Ra, Rb, Rc, R′a, R′b and R′c represents a linear or branched C₈-C₃₀, better still C₁₀-C₂₄, or even C₁₀-C₁₄, alkyl; mention may be made in particular of the dodecyl radical (C₁₂). Preferably, the other radical(s) represent a linear or branched C₁-C₄ alkyl, in particular methyl.

Preferably, in formula (Ib), only one of the radicals Ra, Rb, Rc, R′a, R′b and R′c represents a linear or branched C₈-C₃₀, better still C₁₀-C₂₄, or even C₁₀-C₁₄, alkyl; mention may be made in particular of the dodecyl radical (C₁₂). Preferably, the other radicals represent a linear or branched C₁-C₄ alkyl, in particular methyl.

Even better still, R may be a group chosen from —N+(CH₃)₃, and —N⁺(C₁₂H₂₅)(CH₃)₂, preferably an —N⁺(CH₃)₃, group.

Even better still, R′ may be a group —N⁺(C₁₂H₂₅)(CH₃)₂, Q′⁻.

The aryl radicals preferably denote phenyl, benzyl, naphthyl or anthryl groups.

The percentage of nitrogen can range from 0.1% to 10% by weight relative to the total weight of polymer, preferably from 0.2% to 5% by weight and better still from 0.5% to 3% by weight.

Mention may notably be made of the polymers having the following INCI names:
Polyquaternium-24, such as the product Quatrisoft LM 200®, sold by Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (Cia alkyl), such as the product Crodacel QS®, sold by Croda.

Mention may also be made of the hydroxyethylcelluloses of formula (Ib) wherein R represents a trimethylammonium halide and R′ represents a dimethyldodecylammonium halide; preferentially, R represents trimethylammonium chloride (CH₃)₃N⁺—, Cl⁻ and R′ represents dimethyldodecylammonium chloride (CH₃)₂(C₁₂H₂₅)N⁺, Cl⁻. This type of polymer is known under the INCI name Polyquaternium-67; as commercial products, mention may be made of the Softcat Polymer SL® polymers, such as SL-100, SL-60, SL-30, SL-5 and SX-1300X, from the company Amerchol/Dow Chemical.

More particularly, the polymers of formula (Ib) are those of which the viscosity at 1% by weight in water at 25° C. is between 1000 and 3000 cPs inclusive, preferentially between 2400 and 2800 cPs. Typically, Softcat Polymer SL-5 has a viscosity of approximately 2500 cPs, Softcat Polymer SL-30 has a viscosity of approximately 2600 cPs, Softcat Polymer SL-60 has a viscosity of approximately 2700 cPs and Softcat Polymer SL-100 has a viscosity of approximately 2800 cPs. The percentage of nitrogen of these polymers can range from 0.5% to 5% by weight relative to the total weight of polymer.

Typically, Softcat Polymer SX-1300X has a viscosity of approximately 1300.

More particularly, the cationic cellulose-based polymer is chosen from hydroxyethylcelluloses that have reacted with a trimethylammonium epoxide and a lauryldimethylammonium epoxide (INCI name Polyquaternium-67) and hydroxyethylcelluloses that have reacted with an epoxide substituted with a trimethylammonium group and called Polyquaternium-10.

Preferably, the cationic cellulose-based polymer(s) are chosen from associative cationic cellulose-based polymers. More preferentially, the cationic cellulose-based polymer is the compound having the INCI name Polyquaternium-67.

The total content of the cationic cellulose-based polymer(s), present in the composition according to the invention, preferably ranges from 0.1% to 10% by weight, preferably from 0.3% to 5% by weight, more preferentially from 0.5% to 3% by weight and better still from 0.75% to 2% by weight, relative to the total weight of the composition.

Sulfonic Anionic Polymer

The composition according to the present invention also comprises one or more particular sulfonic anionic polymers.

For the purposes of the present invention, the expression “anionic polymer” denotes any non-silicone polymer containing anionic groups and/or groups that can be ionized into anionic groups and not containing cationic groups and/or groups that can be ionized into cationic groups.

Anionic polymers that may be mentioned include polymers comprising sulfonic groups and having a number-average molecular weight of between 500 and 1 000 000.

For the purposes of the present invention, the term “sulfonic anionic polymer” is intended to mean an anionic polymer comprising in its structure at least one sulfonic group (SO₃H or SO₃⁻).

When the anionic polymer(s) are in salt form, they may be chosen from alkali metal salts such as the sodium or potassium salt and preferably the sodium salt, ammonium salts, amine salts and in particular amino alcohol salts or alkaline-earth metal salts such as the magnesium salts.

Examples of amino alcohol salts that may especially be mentioned include monoethanolamine, diethanolamine and triethanolamine salts, monoisopropanolamine, diisopropanolamine and triisopropanolamine salts, and 2-amino-2-methyl-1-propanol, 2-amino-2-methyl-1,3-propanediol and tris(hydroxymethyl)aminomethane salts.

Use is preferably made of ammonium, alkali metal or alkaline-earth metal salts and in particular of sodium or magnesium salts.
The anionic polymers comprising sulfonic groups can be chosen from:
A′) homopolymers of acrylamidoalkylsulfonic acid or salts thereof.
These polymers may notably be chosen from:
   polyacrylamidosulfonic acids or salts thereof, such as those mentioned in patent U.S. Pat. No. 4,128,631, and more particularly the polyacrylamidoethylpropanesulfonic acid sold under the name Rheocare HSP-1180 by Cognis (INCI name polyacrylamidomethylpropane sulfonic acid), or else optionally neutralized polyacrylamidomethylpropanesulfonic acid, such as the compound sold under the name Hostacerin AMPS by the company Clariant;
      B′) sulfonic polyesters, these polymers being advantageously obtained by polycondensation of at least one dicarboxylic acid, of at least one diol or of a mixture of diol and of diamine, and of at least one difunctional monomer including a sulfonic function. Among these polymers, mention may be made of:
   linear sulfonic polyesters, such as those described in patent applications U.S. Pat. Nos. 3,734,874, 3,779,993, 4,119,680, 4,300,580, 4,973,656, 5,660,816, 5,662,893 and 5,674,479. Such polymers are, for example, the products Eastman® AQ38S Polymer, Eastman® AQ55S Polymer and Eastman® AQ48 Ultra Polymer sold by Eastman Chemical (name: Polyester-5) which are copolymers obtained from diethylene glycol, from 1,4-cyclohexanedimethanol, from isophthalic acid and from salt of sulfoisophthalic acid.
   branched sulfonic polyesters such as those described in patent applications WO 95/18191, WO 97/08261 and WO 97/20899. Such compounds are, for example, the products Eastman® AQ10D Polymer (name: Polyester-13) or Eastman® AQ1350 Polymer which are provided by Eastman Chemical (name: Polyester-13).

Among the anionic polymers comprising sulfonic groups of the family A′, use will more particularly be made of crosslinked or non-crosslinked 2-acrylamido-2-methylpropanesulfonic acid homopolymers and salts thereof, such as in particular Hostacerin AMPS® from Clariant (INCI name Ammonium Polyacryloyldimethyl Taurate).

C′) amphiphilic polymers of at least one acrylamido-2-methylpropanesulfonic acid monomer (AMPS®) and of at least one ethylenically unsaturated hydrophobic comonomer comprising at least one hydrophobic part containing from 7 to 30 carbon atoms.

For the purposes of the invention, the term “amphiphilic polymer” is intended to denote a polymer having amphiphilic properties, i.e. having at least one hydrophilic part and at least one hydrophobic part. Hydrophilic groups and hydrophobic groups are well known to those skilled in the art.

They may be crosslinked or non-crosslinked.

When the amphiphilic polymers according to the invention are crosslinked, the crosslinking agents can be chosen from olefinically polyunsaturated compounds commonly used for crosslinking polymers obtained by radical polymerization, in particular methylenebisacrylamide, allyl methacrylate or trimethylolpropane triacrylate (TMPTA).

The degree of crosslinking may range, for example, from 0.01 mol % to 10 mol % and preferably from 0.2 mol % to 2 mol % relative to the polymer.

The amphiphilic polymers according to the invention may be chosen especially from random amphiphilic AMPS® polymers modified by reaction with a C₆-C₂₂ n-monoalkylamine or di-n-alkylamine, such as those described in patent application WO 00/31154.

An amphiphilic polymer that is suitable for use in the invention may comprise at least one ethylenically unsaturated hydrophilic comonomer chosen, for example, from acrylic acid, methacrylic acid or substituted alkyl derivatives thereof or esters thereof obtained with monoalkylene or polyalkylene glycols, acrylamide, methacrylamide, vinylpyrrolidone, vinylformamide, maleic anhydride, itaconic acid or maleic acid, or mixtures thereof.

An amphiphilic polymer according to the invention may comprise at least one ethylenically unsaturated hydrophobic comonomer.

An amphiphilic polymer that is suitable for use in the invention may comprise at least one hydrophobic part chosen from saturated or unsaturated linear alkyl radicals, for instance n-octyl, n-decyl, n-hexadecyl, n-dodecyl and oleyl, branched alkyl radicals, for instance isostearic, or cyclic alkyl radicals, for instance cyclododecane or adamantane.

An ethylenically unsaturated hydrophobic comonomer of the invention may preferably be chosen from the acrylates or acrylamides of formula (I) below:

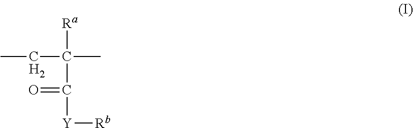

wherein:
R^{a} denotes a hydrogen atom or a linear or branched C₁-C₆ alkyl radical, preferably methyl;
Y denotes 0 or NH;
Rb denotes a hydrophobic radical comprising a fatty chain containing from 7 to 30 carbon atoms, preferably from 7 to 22 and more particularly from 12 to 22 carbon atoms.

The hydrophobic radical Rb is chosen from saturated or unsaturated linear C₇-C₂₂ alkyl radicals (for example n-octyl, n-decyl, n-hexadecyl, n-dodecyl or oleyl), branched alkyl radicals (for example isostearic) or cyclic alkyl radicals (for example cyclododecane or adamantane); C₇-C₁₈ alkylperfluoro radicals (for example the group of formula —(CH₂)₂—(CF₂)₉-CF₃); the cholesteryl radical or a cholesterol ester, for instance cholesteryl hexanoate; aromatic polycyclic groups, for instance naphthalene or pyrene.

Among these radicals, linear and branched alkyl radicals are more particularly preferred.

According to one preferred embodiment of the invention, the hydrophobic radical Rb may also comprise at least one alkylene oxide unit and preferably a polyoxyalkylene chain.

The polyoxyalkylene chain may preferentially be constituted of ethylene oxide units and/or propylene oxide units and even more particularly be constituted solely of ethylene oxide units.

The number of moles of oxyalkylene units may generally range from 1 to 30 mol, more preferentially from 1 to 25 mol and even more preferentially from 3 to 20 mol.

Among these polymers, mention may be made of:
crosslinked or non-crosslinked, neutralized or non-neutralized copolymers comprising from 15% to 60% by weight of AMPS® units and from 40% to 85% by weight of (C₈-C₁₆)alkyl(meth)acrylamide units or of (C₈-C₁₆)alkyl (meth)acrylate units relative to the polymer, such as those described in application EP-A-0 750 899;
terpolymers comprising from 10 mol % to 90 mol % of acrylamide units, from 0.1 mol % to 10 mol % of AMPS® units and from 5 mol % to 80 mol % of n(C₆-C₁₈)alkylacrylamide units relative to the polymer, such as those described in patent U.S. Pat. No. 5,089,578;
non-crosslinked copolymers of partially or completely neutralized AMPS® and of n-dodecyl methacrylate, n-hexadecyl methacrylate or n-octadecyl methacrylate, such as those described in the Morishima articles mentioned above;
crosslinked or non-crosslinked copolymers of partially or completely neutralized AMPS® and of n-dodecylmethacrylamide, such as those described in the Morishima articles mentioned above.

Amphiphilic AMPS® polymers that may also be mentioned include copolymers of totally neutralized AMPS® and of n-dodecyl, n-hexadecyl and/or n-octadecyl methacrylate, and also non-crosslinked and crosslinked copolymers of AMPS® and of n-dodecylmethacrylamide.

Mention will be made more particularly of crosslinked or non-crosslinked amphiphilic AMPS® copolymers constituted:

of 2-acrylamido-2-methylpropanesulfonic acid (AMPS®) units of formula (II) below:

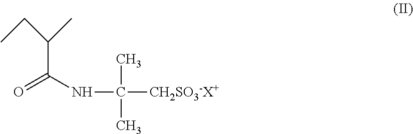

wherein X is a proton, an alkali metal cation, an alkaline-earth metal cation or the ammonium ion;

and of units of following formula (III):

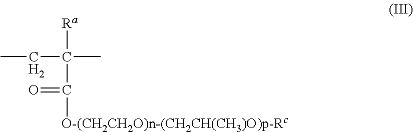

wherein n and p, independently of each other, denote a number of moles and range from 0 to 30, preferably from 1 to 25 and more preferentially from 3 to 20, with the proviso that n+p is less than or equal to 30, preferably less than 25 and even better still less than 20; R^{a} denotes a hydrogen atom or a linear or branched C₁-C₆ alkyl radical, preferably methyl, and R^{c} denotes a linear or branched alkyl containing from 7 to 22 carbon atoms and preferably from 12 to 22 carbon atoms.

In formula (II), the cation X more particularly denotes sodium or ammonium.

The products that will be chosen more particularly are:
those which are non-crosslinked, for which p=0, n=7 or 25, R^{a} denotes a methyl and R^{c} represents a mixture of C₁₂-C₁₄ or C₁₆-C₁₈ alkyl,
those which are crosslinked, for which p=0, n=8 or 25, R^{a} denotes a methyl and R^{c} represents a mixture of C₁₆-C₁₈ alkyl.

These polymers are described and synthesized in application EP 1 069 142.

According to one preferred embodiment, the sulfonic polymers used according to the invention are copolymers of acrylamido-2-methylpropanesulfonic acid and of C₁₂-C₂₂ alkyl (meth)acrylate comprising from 6 to 30 mol of ethylene oxide, and salts thereof.

As a guide, and without this being limiting, mention may notably be made of
a non-crosslinked copolymer of acrylamido-2-methylpropanesulfonic acid and of polyethoxylated C₁₂-C₁₄ alkyl methacrylate comprising 7 mol of ethylene oxide (INCI name: Ammonium Acryloyldimethyltaurate/Laureth-7 Methacrylate Copolymer) sold under the name Aristoflex LNC by the company Clariant;
a crosslinked copolymer of acrylamido-2-methylpropanesulfonic acid and of polyethoxylated stearyl methacrylate comprising 25 mol of ethylene oxide (INCI name: Ammonium Acryloyldimethyltaurate/Steareth-25 Methacrylate Crosspolymer) sold under the name Aristoflex HMS by the company Clariant;
a non-crosslinked copolymer of acrylamido-2-methylpropanesulfonic acid and of polyethoxylated C₁₆/C₁₈ alkyl methacrylate comprising 8 mol of ethylene oxide (INCI name: Ammonium Acryloyldimethyltaurate/Steareth-8 Methacrylate Copolymer) sold under the name Aristoflex SNC by the company Clariant;
a crosslinked copolymer of acrylamido-2-methylpropanesulfonic acid and of polyethoxylated behenyl methacrylate comprising 25 mol of ethylene oxide, crosslinked by trimethylolpropane triacrylate (INCI name: Ammonium Acryloyldimethyltaurate/Beheneth-25 Methacrylate Crosspolymer) sold under the name Aristoflex HMB by the company Clariant; and mixtures thereof.

According to one preferred embodiment, the polymer used according to the invention is a copolymer of AMPS and of a C₁₆-C₁₈ alkyl methacrylate comprising from 6 to 25 mol of ethylene oxide, this copolymer being obtained from AMPS and from methacrylic acid or a methacrylic acid salt and from a polyoxyethylenated C₁₆-C₁₈ alcohol comprising from 6 to 25 mol of ethylene oxide.

According to the invention, the sulfonic anionic polymers are preferably chosen from crosslinked or non-crosslinked 2-acrylamido-2-methylpropanesulfonic acid homopolymers, or salts thereof, and copolymers of acrylamido-2-methylpropanesulfonic acid and of C₁₂-C₂₂ alkyl (meth)acrylate comprising from 6 to 30 mol of ethylene oxide or salts thereof, and mixtures thereof.

The total content of the sulfonic anionic polymer(s), present in the composition according to the invention, preferably ranges from 0.05% to 15% by weight, more preferentially from 0.1% to 10% by weight, even better still from 0.3% to 5% by weight, and better still from 0.5% to 3% by weight, relative to the total weight of the composition.

The weight ratio between the total content of cationic cellulose-based polymer(s) and the total content of sulfonic anionic polymer(s) (PC/PA), present in the composition according to the invention, preferably ranges from 0.1 to 10, preferentially from 0.2 to 5, better still from 0.5 to 3.

The total content of cationic cellulose-based polymer(s) and of sulfonic anionic polymer(s) preferably ranges from 0.1% to 15% by weight, preferentially from 0.5% to 10% by weight, better still from 1% to 5% by weight relative to the total weight of the composition.

Fixing Polymer

The composition according to the present invention also comprises one or more fixing polymers other than the cationic cellulose-based polymers and the sulfonic anionic polymers according to the invention.

For the purposes of the present invention, the term “fixing polymer” is understood to mean a polymer which makes possible the fixing of the shaping of keratin fibers, in particular of the hair.

Preferably, the fixing polymer(s) are chosen from anionic fixing polymers, cationic fixing polymers, amphoteric fixing polymers, nonionic fixing polymers, and mixtures thereof.

The anionic fixing polymers which can be used according to the present invention are polymers comprising groups derived from carboxylic or phosphoric acid and have a number-average molecular weight of between approximately 500 and 5 000 000.

The carboxylic groups are provided by unsaturated mono- or dicarboxylic acid monomers, such as those corresponding to the formula (I):

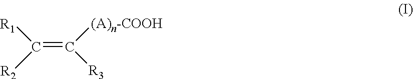

wherein n is an integer from 0 to 10, A₁ denotes a methylene group, optionally connected to the carbon atom of the unsaturated group or to the adjacent methylene group when n is greater than 1, via a heteroatom, such as oxygen or sulfur, R₇ denotes a hydrogen atom or a phenyl or benzyl group, R₈ denotes a hydrogen atom or a lower alkyl or carboxyl group, and R₉ denotes a hydrogen atom, a lower alkyl group or a —CH₂—COOH, phenyl or benzyl group.

In the abovementioned formula, a lower alkyl group preferably denotes a group having from 1 to 4 carbon atoms and in particular the methyl and ethyl groups.

The anionic fixing polymers containing carboxylic groups that are preferred according to the invention are:

A) copolymers of acrylic or methacrylic acid or salts thereof.

Among these polymers, mention may be made of copolymers of acrylic or methacrylic acid with a monoethylenic monomer, such as ethylene, styrene, vinyl esters or acrylic or methacrylic acid esters, optionally grafted to a polyalkylene glycol, such as polyethylene glycol, and optionally crosslinked. Such polymers are described especially in French Patent 1 222 944 and German application No. 2 330 956, the copolymers of this type comprising an optionally N-alkylated and/or N-hydroxyalkylated acrylamide unit in their chain, as are described in particular in Luxembourgian patent applications Nos. 75370 and 75371. Mention may also be made of copolymers of acrylic acid and of C₁-C₄ alkyl methacrylate and terpolymers of vinylpyrrolidone, of acrylic acid and of C₁-C₂₀ alkyl methacrylate, for example lauryl methacrylate, such as that sold by ISP under the name Acrylidone® LM (INCI name: VP/acrylates/lauryl methacrylate copolymer), acrylic acid/ethyl acrylate/N-(t-butyl)acrylamide terpolymers, such as the products Ultrahold® Strong and Ultrahold® 8 sold by BASF (INCI name: Acrylates/t-butylacrylamide copolymer), methacrylic acid/ethyl acrylate/tert-butyl acrylate terpolymers, such as the products sold under the name Luvimer® 100 P or Luvimer® PRO 55 by BASF (INCI name: Acrylates copolymer), copolymers of methacrylic acid and of ethyl acrylate, such as the products sold under the name Luvimer® MAE or Luviflex® Soft by BASF (INCI name: Acrylates copolymer), acrylic acid/butyl acrylate/methyl methacrylate terpolymers, such as the product sold under the name Balance® CR by Akzo Nobel (INCI name: Acrylates copolymer), or the copolymers of methacrylic acid and of methyl methacrylate sold under the name Eudragit® L 100 by Rohm Pharma (INCI name: Acrylates copolymer). Mention may also be made of branched block polymers containing (meth)acrylic acid monomers, such as the product sold under the name Fixate G-100L by Lubrizol (INCI name: AMP-acrylates/allyl methacrylate copolymer);

B) Crotonic acid copolymers, such as those comprising, in their chain, vinyl acetate or propionate units and optionally other monomers, such as allyl or methallyl esters, vinyl ethers or vinyl esters of a linear or branched saturated carboxylic acid having a long hydrocarbon-based chain, such as those comprising at least 5 carbon atoms, it optionally being possible for these polymers to be grafted or crosslinked, or also another monomer which is a vinyl, allyl or methallyl ester of an α- or β-cyclic carboxylic acid. Such polymers are described, inter alia, in French Patents 1 222 944, 1 580 545, 2 265 782, 2 265 781, 1 564 110 and 2 439 798. Commercial products coming within this category are the products Resye 28-2930 and 28-1310 sold by Akzo Nobel (INCI names: VA/crotonates/vinyl decanoate copolymer and VA/crotonates copolymer, respectively). Mention may also be made of the products Luviset® CA 66 sold by the company BASF, Aristoflee A60 sold by the company Clariant (INCI name: VA/crotonates copolymer) and Mexomere PW or PAM sold by the company Chimex (INCI name: VA/vinyl butyl benzoate/crotonates copolymer);

C) copolymers of C₄-C₈ monounsaturated carboxylic acids or anhydrides chosen from:
copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and esters thereof, the anhydride functions of these copolymers optionally being monoesterified or monoamidated. Such polymers are described in particular in U.S. Pat. No. 2,047,398, 2,723,248 and 2,102,113, and GB Patent 839 805. Commercial products are in particular those sold under the names Gantrez® AN or ES by ISP, such as Gantrez® ES 225 (INCI name: Ethyl ester of PVM/MA copolymer) or Gantrez® ES 425L (INCI name: Butyl ester of PVM/MA copolymer).
copolymers comprising (i) one or more maleic, citraconic or itaconic anhydride units and (ii) one or more monomers chosen from allyl or methallyl esters, optionally comprising one or more acrylamide, methacrylamide, α-olefin, acrylic or methacrylic ester, acrylic or methacrylic acid or vinylpyrrolidone groups in their chain, the anhydride functions of these copolymers optionally being monoesterified or monoamidated.

These polymers are, for example, described in Patents FR 2 350 384 and FR 2 357 241;

D) polyacrylamides including carboxylate groups.

According to the invention, the anionic fixing polymers are preferably chosen from acrylic acid copolymers, such as the acrylic acid/ethyl acrylate/N-tert-butylacrylamide terpolymers notably sold under the name Ultrahold® Strong by the company BASF, copolymers derived from crotonic acid, such as the vinyl acetate/vinyl tert-butylbenzoate/crotonic acid terpolymers and the crotonic acid/vinyl acetate/vinyl neododecanoate terpolymers notably sold under the name Resyn 28-2930 by the company Akzo Nobel, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the names Gantrez® ES 425L or ES 225 by the company ISP, the copolymers of methacrylic acid and of ethyl acrylate sold under the name Luvimer® MAE by the company BASF, and the vinyl acetate/crotonic acid copolymers sold under the name Luviset® CA 66 by the company BASF, and the vinyl acetate/crotonic acid copolymers grafted with polyethylene glycol sold under the name Aristoflex® A60 by the company Clariant, the vinylpyrrolidone/acrylic acid/lauryl methacrylate terpolymers sold under the name Acrylidone® LM by the company ISP, the polymer sold under the name Fixate® G-100L by the company Lubrizol, the vinyl acetate/crotonic acid/vinyl p-tert-butylbenzoate copolymers sold under the names Mexomere® PW or PAM by the company Chimex.

The cationic fixing polymers which can be used according to the present invention are preferably chosen from polymers comprising primary, secondary, tertiary and/or quaternary amine groups forming part of the polymer chain or directly connected to the latter, and having a molecular weight of between 500 and approximately 5 000 000 and preferably between 1000 and 3 000 000.

Among these polymers, mention may be made more particularly of the following cationic polymers:
(1) homopolymers or copolymers derived from acrylic or methacrylic esters or amides and including at least one of the units of the following formulae:

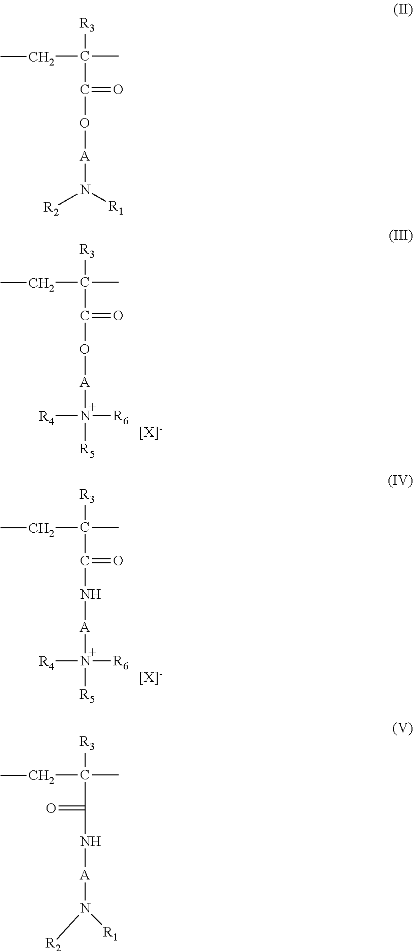

wherein:
R₃ denotes a hydrogen atom or a CH₃ radical;
A is a linear or branched alkyl group comprising from 1 to 6 carbon atoms or a hydroxyalkyl group comprising from 1 to 4 carbon atoms;
R₄, R₅ and R₆, which are identical or different, represent an alkyl group having from 1 to 18 carbon atoms or a benzyl radical;
R₁ and R₂, which are identical or different, each represent a hydrogen atom or an alkyl group having from 1 to 6 carbon atoms; and
X denotes a methosulfate anion or a halide, such as chloride or bromide.

The copolymers of the family (1) additionally comprise one or more units deriving from comonomers which can be chosen from the family of the acrylamides, methacrylamides, diacetone acrylamides, acrylamides and methacrylamides substituted on the nitrogen by lower (C₁-C₄) alkyl groups, groups derived from acrylic or methacrylic acids or their esters, vinyllactams, such as vinylpyrrolidone or vinylcaprolactam, or vinyl esters.

Thus, among these copolymers of the family (1), mention may be made of:
vinylpyrrolidone/dialkylaminoalkyl acrylate or methacrylate copolymers, which may or may not be quaternized, such as the products sold under the Gafquat® name by ISP, such as, for example, Gafquat® 734 or Gafquat® 755 or Gafquat® 755N (INCI name: Polyquaternium-11), or alternatively the products known as Copolymer® 845, 958 and 937 sold by ISP (INCI name: VP/dimethylaminoethyl methacrylate copolymer). These polymers are described in detail in French Patents 2 077 143 and 2 393 573,
polymers comprising a fatty chain and comprising a vinylpyrrolidone unit, such as the products sold under the names Styleze W20L and Styleze W10 by ISP (INCI name: Polyquaternium-55),
dimethylaminoethyl methacrylate/vinylcaprolactam/vinylpyrrolidone terpolymers, such as the products sold under the names Advantage HC 37 or Gaffix® VC 713 by ISP (INCI name: Vinylcaprolactam/VP/dimethylaminoethyl methacrylate copolymer), and
and quaternized vinylpyrrolidone/dimethylaminopropylmethacrylamide copolymers, such as the products sold under the name Gafquat® HS 100 by ISP (INCI name: Polyquaternium-28);

(2) cationic derivatives of guar gum, preferably comprising quaternary ammonium, such as those described in U.S. Pat. No. 3,589,578 and 4 031 307, such as guar gums containing cationic trialkylammonium groups. Such products are sold in particular under the trade names of Jaguar® C13 S, Jaguar® C 15 and Jaguar® C 17 by Rhodia (INCI name: Guar hydroxypropyltrimonium chloride);

(3) quaternary copolymers of vinylpyrrolidone and of vinylimidazole; mention may be made, for example, of vinylpyrrolidone/methylvinylimidazolium chloride copolymers, such as the products sold by BASF under the names Luviquat® FC550 or FC370, Luviquat® Excellence, Luviquat® Style (INCI name: Polyquaternium-16), or of vinylpyrrolidone/vinylimidazolium methosulfate/vinylcaprolactam terpolymers, such as the product Luviquat® Hold sold by BASF (INCI name: Polyquaternium-46);

(4) chitosans or salts thereof; the salts that may be used are in particular the acetate, lactate, glutamate, gluconate or pyrrolidonecarboxylate of chitosan. Mention may be made, among these compounds, of the chitosan pyrrolidonecarboxylate sold under the name Kytamer® PC by Amerchol (INCI name: Chitosan PCA).

The amphoteric fixing polymers that may be used in accordance with the invention may be chosen from polymers including units B and C randomly distributed in the polymer chain, where B denotes a unit derived from a monomer including at least one basic nitrogen atom and C denotes a unit derived from an acidic monomer including one or more carboxylic or sulfonic groups or else B and C can denote groups derived from zwitterionic carboxybetaine or sulfobetaine monomers;

B and C can also denote a cationic polymer chain comprising primary, secondary, tertiary or quaternary amine groups, wherein at least one of the amine groups carries a carboxylic or sulfonic group connected via a hydrocarbon-based group, or else B and C form part of a chain of a polymer comprising an α,β-dicarboxyethylene unit, one of the carboxylic groups of which has been reacted with a polyamine comprising one or more primary or secondary amine groups.

The more particularly preferred amphoteric fixing polymers corresponding to the definition given above are chosen from the following polymers:

(1) copolymers bearing acidic vinyl units and basic vinyl units, such as those resulting from the copolymerization of a monomer derived from a vinyl compound bearing a carboxylic group such as, more particularly, acrylic acid, methacrylic acid, maleic acid, α-chloroacrylic acid, and of a basic monomer derived from a substituted vinyl compound containing at least one basic atom, such as, more particularly, dialkylaminoalkyl methacrylate and acrylate, dialkylaminoalkylmethacrylamide and acrylamide. Such compounds are described in U.S. Pat. No. 3,836,537;

(2) polymers including units deriving:
a) from at least one monomer chosen from acrylamides or methacrylamides substituted on the nitrogen atom with an alkyl group,
b) from at least one acidic comonomer containing one or more reactive carboxylic groups, and
c) from at least one basic comonomer such as esters bearing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides that are more particularly preferred according to the invention are compounds wherein the alkyl groups include from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are more particularly chosen from acrylic acid, methacrylic acid, crotonic acid, itaconic acid, maleic acid and fumaric acid and alkyl monoesters, containing 1 to 4 carbon atoms, of maleic or fumaric acids or anhydrides.

The preferred basic comonomers are aminoethyl, butylaminoethyl, N,N′-dimethylaminoethyl and N-(tert-butyl)aminoethyl methacrylates.

The copolymers of which the INCI name is Octylacrylamide/acrylates/butylaminoethyl methacrylate copolymer, such as the products sold under the names Amphomer®, Amphomer® LV71 or Balance 47 by the company Akzo Nobel, are particularly used;

(3) partially or completely acylated and crosslinked polyaminoamides deriving from polyaminoamides of general formula:

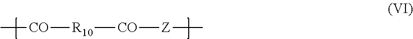

wherein R₁₀ represents a divalent group derived from a saturated dicarboxylic acid, from an aliphatic mono- or dicarboxylic acid bearing an ethylenic double bond, from an ester of a lower alkanol containing from 1 to 6 carbon atoms of these acids, or from a group deriving from the addition of any one of said acids to a bis-primary or bis-secondary amine, and Z denotes a group deriving from a bis-primary, mono- or bis-secondary polyalkylenepolyamine and preferably represents:

a) in the proportions of from 60 mol % to 100 mol %, the group

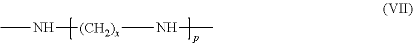

where x=2 and p=2 or 3, or else x=3 and p=2, this group deriving from diethylenetriamine, from triethylenetetramine or from dipropylenetriamine;

b) in the proportions of from 0 mol % to 40 mol %, the group (VII) above wherein x=2 and p=1 and which derives from ethylenediamine, or the group deriving from piperazine:

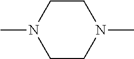

c) in the proportions of from 0 mol % to 20 mol %, the group —NH—(CH₂)₆—NH— deriving from hexamethylenediamine, these polyaminoamides being crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyaminoamide and acylated by the action of acrylic acid, chloroacetic acid or an alkane sultone, or salts thereof.

The saturated carboxylic acids are preferably chosen from acids having from 6 to 10 carbon atoms, such as adipic acid, 2,2,4-trimethyladipic acid and 2,4,4-trimethyladipic acid, terephthalic acid or acids comprising an ethylenic double bond, such as, for example, acrylic acid, methacrylic acid or itaconic acid.

The alkane sultones used in the acylation are preferably propane or butane sultone and the salts of the acylating agents are preferably the sodium or potassium salts;

(4) polymers comprising zwitterionic units of formula:

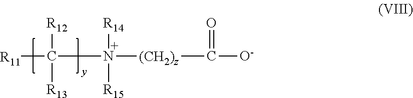

wherein Ru denotes a polymerizable unsaturated group, such as an acrylate, methacrylate, acrylamide or methacrylamide group, y and z represent an integer from 1 to 3, R₁₂ and R₁₃ represent a hydrogen atom or a methyl, ethyl or propyl group, and R₁₄ and R₁₅ represent a hydrogen atom or an alkyl group such that the sum of the carbon atoms in R₁₄ and R₁₅ does not exceed 10.

The polymers comprising such units can also comprise units derived from non-zwitterionic monomers, such as dimethyl- or diethylaminoethyl acrylate or methacrylate or alkyl acrylates or methacrylates, acrylamides or methacrylamides, or vinyl acetate.

Mention may be made, by way of example, of methyl methacrylate/methyl dimethylcarboxymethylammonioethyl methacrylate copolymers, such as the product sold under the name Diaformer Z-301N or Z301W by the company Clariant (INCI name: Acrylates copolymer);

5) polymers derived from chitosan comprising monomer units corresponding to the following formulae:

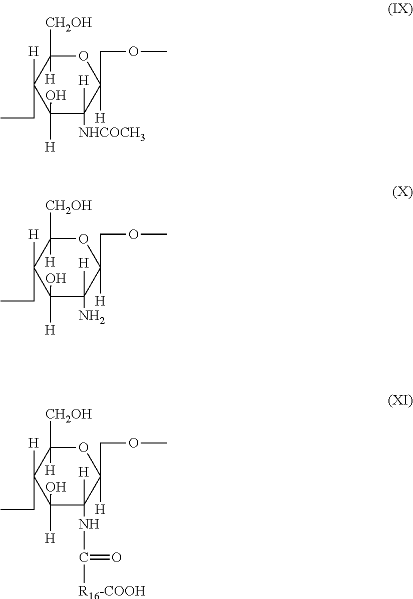

the unit (IX) being present in proportions of between 0% and 30%, the unit (X) in proportions of between 5% and 50% and the unit (XI) in proportions of between 30% and 90%, it being understood that, in this unit (XI), R₁₆ represents a group of formula:

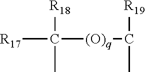

wherein, if q=0, R₁₇, R₁₈ and R₁₉, which are identical or different, each represent a hydrogen atom, a methyl, hydroxyl, acetoxy or amino residue, a monoalkylamine residue or a dialkylamine residue, which are optionally interrupted by one or more nitrogen atoms and/or optionally substituted by one or more amine, hydroxyl, carboxyl, alkylthio or sulfonic groups, or an alkylthio residue wherein the alkyl group carries an amino residue, at least one of the R₁₇, R₁₈ and R₁₉ groups being, in this case, a hydrogen atom;

or, if q=1, R₁₇, R₁₈ and R₁₉ each represent a hydrogen atom, and also the salts formed by these compounds with bases or acids;

(6) polymers containing units corresponding to the general formula (XII) are, for example, described in French Patent 1 400 366:

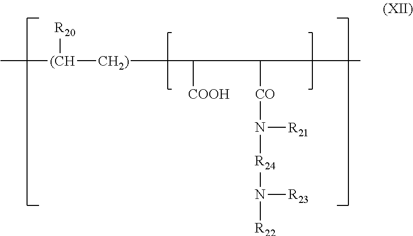

wherein R₂₀ represents a hydrogen atom or a CH₃O, CH₃CH₂O or phenyl group, R₂₁ denotes a hydrogen atom or a lower alkyl group, such as methyl or ethyl, R₂₂ denotes a hydrogen atom or a lower C₁-C₆ alkyl group, such as methyl or ethyl, and R₂₃ denotes a lower C₁-C₆ alkyl group, such as methyl or ethyl, or a group corresponding to the formula: —R₂₄—N(R₂₂)₂, R₂₄ representing a —CH₂—CH₂—, —CH₂—CH₂—CH₂— or —CH₂—CH(CH₃)— group and R₂₂ having the meanings mentioned above;

(7) polymers derived from the N-carboxyalkylation of chitosan, such as N-carboxymethyl chitosan or N-carboxybutyl chitosan, for instance the product sold under the name Chitoglycan by the company Sinerga SPA (INCI name Carboxymethyl chitosan);

(8) amphoteric polymers of the -D-X-D-X type chosen from:

a) polymers obtained by reaction of chloroacetic acid or sodium chloroacetate with compounds comprising at least one unit of formula:

-D-X-D-X-D- (XIII)

where D denotes a group

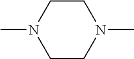

and X denotes the symbol E or E′, where E and E′, which may be identical or different, denote a divalent group that is an alkylene group with a straight or branched chain including up to 7 carbon atoms in the main chain, which is unsubstituted or substituted with hydroxyl groups and which may include, in addition to oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups;

b) polymers of formula: -D-X-D-X— (XIII′)

where D denotes a group

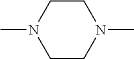

and X denotes the symbol E or E′ and at least once E′; E having the meaning given above and E′ being a divalent group that is an alkylene group with a straight or branched chain containing up to 7 carbon atoms in the main chain, which is unsubstituted or substituted with one or more hydroxyl groups and which includes one or more nitrogen atoms, the nitrogen atom being substituted with an alkyl chain that is optionally interrupted with an oxygen atom and which necessarily includes one or more carboxyl functions or one or more hydroxyl functions betainized by reaction with chloroacetic acid or sodium chloroacetate;

(9) (C₁-C₅)Alkyl vinyl ether/maleic anhydride copolymers partially modified by semiamidation with an N,N-dialkylaminoalkylamine, such as N,N-dimethylaminopropylamine, or by semiesterification with an N,N-dialkylaminoalkanol. These copolymers can also comprise other vinyl comonomers, such as vinylcaprolactam.

Mention will be made, among the amphoteric fixing polymers mentioned above which are the most particularly preferred according to the invention, of those of family (3), such as the copolymers for which the INCI name is Octylacrylamide/acrylates/butylaminoethyl methacrylate copolymer, such as the products sold under the name Amphomer®, Amphomer® LV 71 or Balance® 47 by Akzo Nobel, and those of family (4), such as methyl methacrylate/methyl dimethylcarboxymethylammonioethyl methacrylate copolymers, sold, for example, under the name Diaformer Z-301N or Z-301W by Clariant.

Preferably, the fixing polymer(s) are chosen from nonionic fixing polymers and mixtures thereof, and more preferentially from:
polyalkyloxazolines;
vinyl acetate homopolymers;
vinyl acetate copolymers, for instance copolymers of vinyl acetate and of acrylic ester, copolymers of vinyl acetate and of ethylene, or copolymers of vinyl acetate and of maleic ester, for example of dibutyl maleate;
acrylic ester homopolymers and copolymers, such as, for example, copolymers of alkyl acrylates and of alkyl methacrylates, such as the products provided by Röhm GmbH under the name Eudragit NE 30 D (INCI name: Acrylates copolymer);
copolymers of acrylonitrile and of a nonionic monomer chosen, for example, from butadiene and alkyl (meth)acrylates;
styrene homopolymers;
styrene copolymers, for instance copolymers of styrene, of alkyl acrylate and of alkyl methacrylate; copolymers of styrene and of butadiene; or copolymers of styrene, of butadiene and of vinylpyridine;
polyamides;
vinyllactam homopolymers, such as vinylpyrrolidone homopolymers, for example sold under the names Luviskol® K30 powder by BASF or PVP K30L or K60 solution or K90 by ISP, or such as the polyvinylcaprolactam sold under the name Luviskol Plus by BASF (INCI name: PVP);
vinyllactam copolymers, such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVP/VA® S630L, E735, E635 and W735 by the company ISP, Luviskol® VA 73, VA 64 and VA 37 by the company BASF (INCI name: VP/VA copolymer); and vinylpyrrolidone/methacrylamide/vinylimidazole terpolymers, for instance the product sold under the name Luviset® Clear by the company BASF (INCI name: VP/methacrylamide/vinyl imidazole copolymer);
and mixtures thereof.

The alkyl groups of the nonionic polymers mentioned above preferably contain from 1 to 6 carbon atoms.

Use may also be made, as fixing polymers, of functionalized or nonfunctionalized and silicone or non-silicone cationic, nonionic, anionic or amphoteric polyurethanes, or mixtures thereof.

The polyurethanes particularly targeted by the present invention are those described in applications EP 0 751 162, EP 0 637 600, EP 0 648 485 and FR 2 743 297, of which the applicant is the proprietor, and also in applications EP 0 656 021 and WO 94/03510 from the company BASF and EP 0 619 111 from the company National Starch.

Mention may be made, as polyurethanes particularly highly suitable in the present invention, of the products sold under the names Luviset® PUR and Luviset® Si PUR by BASF (INCI names: Polyurethane-1 and Polyurethane-6 respectively).

Preferably, the fixing polymer(s) are chosen from nonionic fixing polymers and mixtures thereof, more preferentially from vinyllactam homopolymers, such as vinylpyrrolidone homopolymers or polyvinylcaprolactam, and vinyllactam copolymers, such as a poly(vinylpyrrolidone/vinyllactam) copolymer, poly(vinylpyrrolidone/vinyl acetate) copolymers or poly(vinylpyrrolidone/vinyl acetate/vinyl propionate) terpolymers, and mixtures thereof.

Preferentially, the fixing polymer(s) are chosen from vinyllactam homopolymers and vinyllactam copolymers, and mixtures thereof.

Advantageously, the fixing polymer(s) are chosen from vinyllactam homopolymers, such as vinylpyrrolidone homopolymers and polyvinylcaprolactam, and mixtures thereof.

The total amount of the fixing polymer(s), present in the composition according to the invention, preferably ranges from 0.1% to 20% by weight, more preferentially from 0.5% to 15% by weight and better still from 1% to 12% by weight, relative to the total weight of the composition.

Preferably, the composition is free of silicone. The expression “free of silicone” means that the composition comprises less than 0.1% by weight of silicone relative to the total weight of the composition, and preferably does not comprise silicone (0%).

Preferably, the composition according to the present invention is aqueous. The water is present in a content generally of greater than or equal to 30% by weight relative to the total weight of the composition.

Preferably, the water content present in the composition of the invention ranges from 30% to 98% by weight, preferably from 50% to 95% by weight and more preferentially from 65% to 95% by weight, relative to the total weight of the composition.

The composition according to the present invention may optionally comprise one or more organic solvents, or mixtures thereof.

Examples of organic solvents that may be mentioned include linear or branched C₂ to C₄ alkanols, such as ethanol and isopropanol; glycerol; polyols and polyol ethers, for instance 2-butoxyethanol, propylene glycol, hexylene glycol, dipropylene glycol, propylene glycol monomethyl ether, diethylene glycol monomethyl ether and monoethyl ether, and also aromatic alcohols or ethers, for instance benzyl alcohol or phenoxyethanol, and mixtures thereof.

The pH of the composition according to the invention generally ranges from 1.5 to 12, preferably from 2 to 7, preferentially from 2.5 to 6.5 and better still from 3 to 6.

The pH of the composition may be adjusted to the desired value by means of basifying agents or acidifying agents that are customarily used. Among the basifying agents, examples that may be mentioned include aqueous ammonia, alkanolamines, and mineral or organic hydroxides. Among the acidifying agents, examples that may be mentioned include mineral or organic acids, for instance hydrochloric acid, orthophosphoric acid or sulfuric acid, carboxylic acids, for instance acetic acid, tartaric acid, citric acid and lactic acid, and sulfonic acids.

The composition according to the present invention may also optionally comprise one or more additives, different than the compounds of the invention and among which mention may be made of nonionic, cationic, anionic, amphoteric or zwitterionic surfactants and mixtures thereof, fatty substances, thickeners, amphoteric polymers, antidandruff agents, anti-seborrhoea agents, agents for preventing hair loss and/or for promoting hair regrowth, vitamins and provitamins including panthenol, sunscreens, mineral or organic pigments, sequestrants, plasticizers, solubilizers, acidifying agents, opacifiers or nacreous agents, antioxidants, hydroxy acids, fragrances, preserving agents, pigments and ceramides.

According to the invention, the composition also comprises one or more additional salts other than the compounds of the invention.

For the purposes of the present invention, the term “additional salts” is intended to mean alkali metal salts or alkaline-earth metal salts, such as sodium chloride, magnesium chloride, CaCl₂); organic acid salts such as sodium citrate, sodium lactate, sodium acetate, and mixtures thereof; more preferentially alkali metal salts and in particular sodium chloride.

Of course, those skilled in the art will take care to choose this or these optional additional compounds so that the advantageous properties intrinsically associated with the composition according to the invention are not, or not substantially, detrimentally affected by the envisioned addition(s).

The above additives can generally be present in an amount of, for each of them, between 0% and 20% by weight, relative to the total weight of the composition.

The present invention also relates to a cosmetic treatment process, preferably a process for the treatment of human keratin fibers such as the hair, notably a process for styling, i.e. fixing and/or shaping, keratin fibers, in particular human keratin fibers such as the hair, comprising the application to said keratin fibers of a composition as defined above.

The shaping process according to the invention can be carried out on dry or wet keratin fibers, preferably wet.

On conclusion of the process, the keratin fibers are advantageously rinsed with water. They may optionally be washed with a shampoo, followed by rinsing with water, before being dried or left to dry.

In one preferred embodiment of the invention, the process comprises:

(i) a step of applying to the keratin fibers a composition comprising:
(a) one or more cationic cellulose-based polymers,
(b) one or more sulfonic anionic polymers according to the invention, and
(c) one or more fixing polymers other than the polymers (a) and (b), followed by:

(ii) a step of rinsing the keratin fibers, preferably with water,

(iii) optionally a step of shaping the keratin fibers.

After rinsing with water, the hair is shaped, notably with the fingers or a comb or a brush, and then dried. The drying step may be performed at a temperature ranging from 20° C. to 80° C. In practice, the drying step may take place either at ambient temperature or using a hairdryer.

The present invention finally relates to the use of a composition as described above, for the cosmetic treatment of, and notably for styling, keratin fibers, in particular human keratin fibers such as the hair.

More particularly, the present invention relates to the use of a composition as described above, for styling the hair, that is to say shaping and/or fixing (holding) the hairstyle.

The examples that follow serve to illustrate the invention without, however, being limiting in nature.

### EXAMPLES

### Example 1

Compositions (A1), (A2), (A3), (A4), (A5), (A6) and (A7) were prepared using the ingredients of which the contents, expressed as weight percentage of active material, relative to the total weight of each composition, are mentioned in the table below.

TABLE 1

| | A1 | | | A4 | | A6 | |
|---|---|---|---|---|---|---|---|
| | (inv) | A2 | A3 | (inv) | A5 | (inv) | A7 |
| Ammonium | 0.84 | 0.84− | | 0.84 | | 0.84 | |
| polyacryloyldimethyltaurate | | | | | | | |
| (Hostacerin AMPS ®) | | | | | | | |
| Polyquaternium-67 | 1.16 | | 1.16 | | | | |
| (Softcat polymer SL-100) | | | | | | | |
| Polyquaternium-10 | | | | 1.16 | 1.16 | | |
| Polyquaternium-4 | | | | | | 1.16 | 1.16 |
| Sodium chloride | 2 | 2 | 2 | 2 | 2 | 2 | 2 |
| Vinyl | 3 | 3 | 3 | 3 | 3 | 3 | 3 |
| acetate/vinylpyrrolidone | | | | | | | |
| copolymer (Luviskol VA 64 | | | | | | | |
| from BASF) | | | | | | | |
| pH Agent, qs pH | 3.5 | 3.5 | 3.5 | 3.5 | 3.5 | 3.5 | 3.5 |
| Water qs | 100 g | 100 g | 100 g | 100 g | 100 g | 100 g | 100 g |

Each of compositions (A1) to (A7) thus obtained was applied to locks of natural hair weighing 2.7 g, prewashed with a DOP shampoo, in a proportion of 1 g of composition per lock.

After a leave-on time of approximately 15 seconds, the locks of hair were rinsed with water for 15 seconds. The locks are then toweled dry and then deposited flat and left to air dry.

At the outcome of this treatment, the fixing and hold of the shape were evaluated on dry hair, by 6 trained experts:
The fixing was evaluated by exerting a pressure on the lock between two fingers of a hand: the more difficult it is to fold the lock of hair, the stronger the fixing.
The hold was evaluated by subjecting the lock to 3 passes of the fingers from the root to the end: the more the lock retains its initial structure after the passing of the fingers, the greater the hold.

These two parameters are graded from 0 to 5. The minimum limit of these two parameters is acquired after wetting a non-treated lock with water: the fixing and also the hold are zero. The maximum limit is obtained after application to a lock of the Vivelle Dop Concrete Force fixing styling gel: its fixing and hold parameters are each 5 out of 5.

Results

TABLE 2

| | A1 | | | A4 | | A6 | |
|---|---|---|---|---|---|---|---|
| | (inv) | A2 | A3 | (inv) | A5 | (inv) | A7 |
| Fixing | 2.5 | 1 | 1 | 3 | 0.5 | 4 | 1.5 |
| Hold of the | 2 | 1 | 1.5 | 3 | 0.5 | 3 | 1 |
| fixing | | | | | | | |

The locks treated with the compositions according to the invention exhibit good fixing and good hold of the fixing, greater than those of the compositions comprising just one of the two polymers.

Furthermore, the locks have a smooth feel and are not tacky.

### Example 2

Compositions (B1), (B2) and (B3) were prepared using the ingredients of which the contents, expressed as weight percentage of active material, relative to the total weight of each composition, are mentioned in the table below.

TABLE 3

| | B1 | | |
|---|---|---|---|
| | (inv) | B2 | B3 |
| Ammonium | 0.84 | 0.84- | |
| acryoyldimethyltaurate/steareth-8 | | | |
| methacrylate copolymer | | | |
| (Aristoflex SNC from Clariant) | | | |
| Polyquaternium-10 | 1.16 | | 1.16 |
| Sodium chloride | 2 | 2 | 2 |
| Vinyl acetate/vinylpyrrolidone | 3 | 3 | 3 |
| copolymer (Luviskol VA 64 from | | | |
| BASF) | | | |
| pH Agent qs | 3.5 | 3.5 | 3.5 |
| Water qs | 100 g | 100 g | 100 g |

Each of compositions (B1) to (B3) thus obtained was applied to locks of natural hair weighing 2.7 g, prewashed with a DOP shampoo, in a proportion of 1 g of composition per lock.

The locks of hair were then rinsed with water for 15 seconds. The locks are then toweled dry and then deposited flat and left to air dry.

At the outcome of this treatment, the fixing and hold of the shape were evaluated on dry hair, by 6 trained experts:
The fixing was evaluated by exerting a pressure on the lock between two fingers of a hand: the more difficult it is to fold the lock of hair, the stronger the fixing.
The hold was evaluated by subjecting the lock to 3 passes of the fingers from the root to the end: the more the lock retains its initial structure after the passing of the fingers, the greater the hold.

These two parameters are graded from 0 to 5. The minimum limit of these two parameters is acquired after wetting a non-treated lock with water: the fixing and also the hold are zero. The maximum limit is obtained after application to a lock of the Vivelle Dop Concrete Force fixing styling gel: its fixing and hold parameters are each 5 out of 5.

Results

TABLE 4

| | A1 (inv) | A2 | A3 |
|---|---|---|---|
| Fixing | 2 | 0.5 | 1.5 |
| Hold of the | 2 | 0.5 | 1 |
| fixing | | | |

The locks treated with the compositions according to the invention exhibit good fixing and good hold of the fixing, greater than those of the compositions comprising just one of the two polymers.

1-15. (canceled)

16. A composition comprising:
a) one or more cationic cellulose-based polymers,
b) one or more sulfonic anionic polymers chosen from:
homopolymers of acrylamidoalkylsulfonic acid or salts thereof,
sulfonic polyesters or salts thereof, or
amphiphilic polymers of at least one acrylamido-2-methylpropanesulfonic acid monomer and of at least one ethylenically unsaturated hydrophobic comonomer comprising at least one hydrophobic part containing from 7 to 30 carbon atoms, and
c) one or more fixing polymers other than the polymers (a) and the polymers (b).

17. The composition of claim 16, wherein one or more of the cationic cellulose-based polymers are chosen from cellulose ethers comprising quaternary ammonium groups, cationic cellulose copolymers, or celluloses grafted with a water-soluble quaternary ammonium monomer.

18. The composition of claim 16, wherein one or more of the cationic cellulose-based polymers are chosen from polyquaternium-10, polyquaternium-67, or a mixture thereof.

19. The composition of claim 16, wherein one or more of the cationic cellulose-based polymers are chosen from associative cationic cellulose-based polymers.

20. The composition of claim 16, wherein the total amount of cationic cellulose-based polymers ranges from 0.1% to 10% by weight, relative to the total weight of the composition.

21. The composition of claim 16, wherein one or more of the sulfonic anionic polymers are chosen from crosslinked 2-acrylamido-2-methylpropanesulfonic acid homopolymers, crosslinked copolymers of acrylamido-2-methylpropanesulfonic acid, non-crosslinked 2-acrylamido-2-methylpropanesulfonic acid homopolymers, non-crosslinked copolymers of acrylamido-2-methylpropanesulfonic acid, C₁₂-C₂₂ alkyl (meth)acrylate comprising from 6 to 30 moles of ethylene oxide, salts thereof, or mixtures thereof.

22. The composition of claim 16, wherein the total amount of sulfonic anionic polymers ranges from 0.05% to 15% by weight, relative to the total weight of the composition.

23. The composition of claim 16, wherein one or more of the fixing polymers are chosen from anionic fixing polymers, cationic fixing polymers, amphoteric fixing polymers, nonionic fixing polymers, or mixtures thereof.

24. The composition of claim 16, wherein one or more of the fixing polymers are chosen from nonionic fixing polymers, vinyl acetate homopolymers, vinyl acetate copolymers, acrylic ester homopolymers, acrylic ester copolymers, copolymers of acrylonitrile and of a nonionic monomer, styrene homopolymers, styrene copolymers, polyamides, vinyllactam homopolymers, vinyllactam copolymers, or mixtures thereof.

25. The composition of claim 16, wherein the total amount of fixing polymers ranges from 0.1% to 20% by weight, relative to the total weight of the composition.

26. The composition of claim 16, wherein a weight ratio of a total amount by weight of cationic cellulose-based polymers and a total amount by weight of sulfonic anionic polymers present in the composition ranges from 0.1 to 10.

27. The composition of claim 16, wherein the composition is aqueous.

28. The composition of claim 16, further comprising water in an amount ranging from 30% to 98% by weight, relative to the total weight of the composition.

29. A process of treating keratin fibers comprising applying to the keratin fibers a composition comprising:
a) one or more cationic cellulose-based polymers,
b) one or more sulfonic anionic polymers chosen from:
homopolymers of acrylamidoalkylsulfonic acid or salts thereof,
sulfonic polyesters or salts thereof, or
amphiphilic polymers of at least one acrylamido-2-methylpropanesulfonic acid monomer and of at least one ethylenically unsaturated hydrophobic comonomer comprising at least one hydrophobic part containing from 7 to 30 carbon atoms, and
c) one or more fixing polymers other than the polymers (a) and the polymers (b).

30. A method for styling keratin fibers comprising applying to the keratin fibers a composition comprising:
a) one or more cationic cellulose-based polymers,
b) one or more sulfonic anionic polymers chosen from:
homopolymers of acrylamidoalkylsulfonic acid or salts thereof,
sulfonic polyesters or salts thereof, or
amphiphilic polymers of at least one acrylamido-2-methylpropanesulfonic acid monomer and of at least one ethylenically unsaturated hydrophobic comonomer comprising at least one hydrophobic part containing from 7 to 30 carbon atoms, and
c) one or more fixing polymers other than the polymers (a) and the polymers (b).

31. The method of claim 30, wherein one or more of the cationic cellulose-based polymers are chosen from cellulose ethers comprising quaternary ammonium groups, cationic cellulose copolymers, or celluloses grafted with a water-soluble quaternary ammonium monomer.

32. The method of claim 30, wherein the total amount of cationic cellulose-based polymers ranges from 0.1% to 10% by weight, relative to the total weight of the composition.

33. The method of claim 30, wherein the total amount of sulfonic anionic polymers ranges from 0.05% to 15% by weight, relative to the total weight of the composition.

34. The method of claim 30, wherein one or more of the fixing polymers are chosen from nonionic fixing polymers, vinyl acetate homopolymers, vinyl acetate copolymers, acrylic ester homopolymers, acrylic ester copolymers, copolymers of acrylonitrile and of a nonionic monomer, styrene homopolymers, styrene copolymers, polyamides, vinyllactam homopolymers, vinyllactam copolymers, or mixtures thereof.

35. The method of claim 30, wherein the total amount of fixing polymers ranges from 0.1% to 20% by weight, relative to the total weight of the composition.
